(19) United States
(12) Patent Application Publication
(10) Pub. No.: US 2011/0031430 A1
(43) Pub. Date: Feb. 10, 2011

(54) SYNTHESIS OF WATER-SOLUBLE ORGANIC NANOPARTICLES AS EPR STANDARD

(75) Inventors: Y. CHARLES CAO, Gainesville, FL (US); Ou Chen, Gainesville, FL (US); Alexander Angerhofer, Gainesville, FL (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: UNIVERSITY OF FLORIDA RESEARCH FOUNDATION INC., Gainesville, FL (US)

(21) Appl. No.: 12/850,088

(22) Filed: Aug. 4, 2010

Related U.S. Application Data
(60) Provisional application No. 61/232,299, filed on Aug. 7, 2009.

(51) Int. Cl. H01F 1/00; C07C 241/02; B32B 5/16

(52) U.S. Cl. 252/62.51 R; 564/314; 428/402; 428/402.24; 977/773; 977/838

(57) ABSTRACT

Novel water soluble paramagnetic organic nanoparticles are formed by a novel method where an organic solution of an organic compound is injected into water under agitation that is maintained for a desired period of time for nanoparticle growth followed by termination of the growth by the addition of an aqueous surfactant solution. The size of the nanoparticles depends on the time between injection and addition of the surfactant solution. In embodiments of the invention, the water soluble paramagnetic organic nanoparticles can be DPPH nanoparticles, DPPH nanoparticles doped with DPPH-H, or core/shell nanoparticles where a DPPH core is covered by a DPPH-H shell.

### CROSS-REFERENCE TO A RELATED APPLICATION

The present application claims the benefit of U.S. Provisional Application Ser. No. 61/232,299, filed Aug. 7, 2009, which is hereby incorporated by reference herein in its entirety, including any figures, tables, or drawings.

### GOVERNMENT SUPPORT

The subject invention was made with government support under a research project supported by National Science Foundation, Contract No. DMR-0645520 and the Office of Naval Research, Contract No. N00014-06-1-0911. The government has certain rights to this invention.

### BACKGROUND OF INVENTION

The discovery of size-dependent properties in inorganic colloidal nanoparticles (NPs) has stimulated research efforts to develop synthetic methods for making NPs of small organic molecule building blocks. Small-molecule organic NPs are formed through non-covalent intermolecular interactions such as π-π interactions, van der Waals forces, hydrogen bonds, and solvophobic interactions. To date, some organic NPs have been synthesized using small molecules that possess a rigid π-system. These organic NPs exhibit size-dependent optical properties that may be exploited as a new class of functional materials and potentially can be used for optoelectronics. Fully organic paramagnetic NPs have not been prepared, even though such nanoparticles should be useful in various magnetic resonance and image technologies.

Measurements using electron paramagnetic resonance (EPR) spectroscopy normally requires a standard field marker and a primary spin-concentration standard. 2,2′-diphenyl-1-picrylhydrazyl (DPPH), a stable organic radical with unique paramagnetic properties, is commonly employed as a marker in solid phase or organic solutions. Because of its narrow paramagnetic resonance, DPPH is used as the standard field marker for g-factor determination and for magnetic scan calibration for low- and high-field EPR measurements. DPPH is also used as a primary spin-concentration standard in quantitative EPR spectrometry for the determination of free radical concentration in various samples. Unfortunately, the low solubility of DPPH in water limits its applications in aqueous solutions. To overcome this limitation, Tamano, et al., Macromol. Rapid Commun. 2006, 27, 1764 reports an approach where DPPH is stabilized in aqueous solutions by encapsulation into aggregates of amphiphilic block copolymers. The DPPH-containing polymer aggregates exhibit single-line EPR spectra, but their linewidths are about 5.0-15 G (Gauss), making these aggregates unsuitable for use as EPR standards.

### BRIEF SUMMARY

Embodiments of the invention are directed to water soluble paramagnetic nanoparticles (NPs), aqueous electron paramagnetic resonance (EPR) standards comprised of the water soluble paramagnetic NPs, and methods for their preparation. The NPs can have a diameter of 5 to 1,000 nm. The paramagnetic NPs comprise stable radicals, such as 2,2′-diphenyl-1-picrylhydrazyl (DPPH). Additionally diamagnetic equivalents to the stable radicals, for example 2, 2′-diphenyl-1-picrylhydrazine (DPPH-H) with DPPH, can be included as a dopant or a shell material in the NPs. In embodiments of the invention, the NPs are amorphous materials. EPR standards for use in aqueous solutions comprising these water soluble paramagnetic nanoparticles (NPs) exhibit narrow line widths, for example, less than 1.80, 1.50 or even 1.18 G.

A method for preparation of a water soluble paramagnetic NPs comprises providing an organic solution comprising a paramagnetic organic compound and a water miscible organic solvent, injecting the organic solution into agitated water to form a growth solution, in which NPs grow in size until an aqueous surfactant solution is injected into the growth solution to fix the NPs size, from which the NPs can be isolated. For example DPPH in tetrahydrofuran (THF) can be added to rapidly stirred water, to which the surfactant gelatin can be added after a period of 1 to 120 minutes to fix the size of DPPH NPs at about 5 to about 1,000 nm. These DPPH nanoparticles can be isolated by centrifugation. In one embodiment of the invention, a diamagnetic compound, such as DPPH-H, can be included in the organic solution containing the paramagnetic compound, such as DPPH, to form doped paramagnetic NPs, such as DPPH doped with DPPH-H. In another embodiment of the invention after formation of NPs comprising a paramagnetic compound, a reducing agent can be included to form a shell of a diamagnetic equivalent of the paramagnetic compound to yield a core/shell NP. For example a DPPH nanoparticle can be reduced, for example with the reducing agent 2,5-dihydroxy-1,4-benzoquinone, on its surface to form a DPPH-H shell on a DPPH core.

### BRIEF DESCRIPTION OF DRAWINGS

FIG. 1 shows TEM images of DPPH NPs of (a) 90 nm, (b) 200 nm, and (c) 310 nm, where the scale bars indicate 500 nm in accordance with an embodiment of the subject invention.

FIG. 2 shows an electron diffraction (ED) pattern of 250 nm DPPH nanoparticles in accordance with embodiments of the invention.

FIG. 3 shows absorption spectra of DPPH in THF solution, DPPH nanoparticles with a diameter of 90 nm, 170 nm, 250 nm, 310 nm in accordance with an embodiment of the invention.

FIG. 4 shows plots of DPPH nanoparticle diameter as a function of: (a) the maximum of absorption band I (UV band) and (b) absorption band II (Vis band) of DPPH in accordance with embodiments of the invention.

FIG. 5 shows an EPR spectrum of a 250-nm DPPH NPs (taken at 9.5 GHz and 298 K) in accordance with an embodiment of the invention.

FIG. 6 shows a plot of DPPH NP's EPR linewidth (LW) as a function of NPs diameter in accordance with embodiments of the invention.

FIG. 7 shows transmission electron microscope (TEM) images of (a) DPPH nanoparticles; (b) DPPH/DPPH-H core/shell nanoparticles; (c) DPPH-H doped DPPH nanoparticles where the scale bars are in 1 μm showing all nanoparticles have a diameter of about 180 nm with a relative standard deviation of 14%, in accordance with embodiments of the invention.

FIG. 8 shows a composite absorption spectra of DPPH NPs (solid line), core/shell NPs) (dashed line), and DPPH-H doped NPs (dotted line) in accordance with embodiments of the invention.

FIG. 9 shows EPR spectra of DPPH NPs (top), DPPH/DPPH-H core/shell NPs (middle) and DPPH-H doped DPPH NPs (bottom) in accordance with embodiments of the invention.

FIG. 10 shows: (a) Absorption spectra of DPPH nanoparticles (˜300 nm in diameter) at various pH (3.0 to 10.0); (b) corresponding electron paramagnetic resonance (EPR) spectra; (c) EPR linewidth from (b) as a function of pH; and (d) g-factor from (b) as a function of pH where the concentration of DPPH nanoparticles was constant with the pH of nanoparticle solution adjusted using standard pH buffers or HCl solutions where the final pH value determined by pH-indicator strips (colorpHast®) in accordance with embodiments of the invention.

FIG. 11 shows a plot of DPPH nanoparticle size in diameter as a function of growth time in accordance with embodiments of the invention.

FIG. 12 is a chemical equation for the synthesis of 2,2′-diphenyl-1-picrylhydrazine (DPPH-H) in accordance with an embodiment of the invention.

FIG. 13 is an illustration of the synthesis of a DPPH/DPPH-H core/shell nanoparticle in accordance with an embodiment of the invention.

FIG. 14 shows the EPR spectrum of DPPH nanoparticles made using low conductivity deionized (DI) water with an EPR line width of 1.18G in accordance with an embodiment of the invention.

### DETAILED DISCLOSURE

Embodiments of the present invention are directed to water soluble paramagnetic organic nanoparticles (NPs), a method for the preparation of organic NPs, and the use of such. NPs as water soluble electron paramagnetic resonance (EPR) spectroscopy standards. In one embodiment of the invention the organic NPs comprise 2,2′-diphenyl-1-picrylhydrazyl (DPPH). The novel method involves a colloidal synthesis approach that yields stable, water-soluble DPPH NPs that exhibit single-line EPR spectra with linewidths of about 1.50-1.80 G, which are better, equal or close to the narrowest linewidth (1.5 G) of the common DPPH standard in a form of water-insoluble microcrystals. Alternatively, the method can be adapted to yield NPs of radicals other than DPPH including: triphenylmethyl radical, polychlorinated triphenylmethyl radicals, tris(2,6-dimethoxyphenyl)methyl radical, phenalenyl and related radicals, and cyclopentadienyl radicals.

The colloidal synthesis of water-soluble NPs, for example, DPPH NPs, according to an embodiment of the invention involves a modified reprecipitation method. The nucleation of organic NPs is initiated by a sudden introduction of solvophobic interactions between molecular building blocks (i.e., small-molecule precursors) and their surrounding solvent molecules, which is achieved by the addition of a poor solvent (e.g., water) for the molecular building blocks. Subsequent nanoparticle growth cannot be terminated simply by a temperature-quenching process, like those used in advanced high-temperature syntheses of inorganic nanocrystal, because the reprecipitation synthesis is carried out around room temperature. The lack of a viable quenching process has limited the preparation of size-controlled organic NPs. In the present method, quenching was carried out in a manner where gelatin, a common surfactant for organic NPs, can be introduced to rapidly terminate the growth of NPs, for example, DPPH NPs, in water. In this manner size control for DPPH NPs can be achieved by the injection of a gelatin solution during particle growth at a determined time. Other reported methods for making organic small-molecule NPs control the final particle size by control of the concentrations of precursors and of surfactant molecules in the precipitating mixture. In contrast, the synthesis method according to an embodiment of the invention controls the final size of organic NPs by choosing the particle-growth time and rapidly quenching the particle growth by rapid addition of the surfactant. Nanoparticles of about 50 to about 1,000 nm can be prepared according to embodiments of the invention. Solvents that can be used in embodiments of the method include tetrahydrofuran (THF), methanol, ethanol, acetonitrile, dimethylformamide, and dimethyl sulfoxide. Surfactants other than gelatin that can be employed in other embodiments of the invention include: anionic surfactants such as sodium dodecyl sulfate (SDS), ammonium lauryl sulfate, other alkyl sulfate salts, sodium laureth sulfate (also known as sodium lauryl ether sulfate: SLES), or alkyl benzene sulfonate; cationic surfactants such as alkyltrimethylammonium salts, cetylpyridinium chloride (CPC), polyethoxylated tallow amine (POEA), benzalkonium chloride (BAC), and benzethonium chloride (BZT); zwitterionic surfactant such as dodecyl betaine, dodecyl dimethylamine oxide, cocamidopropyl betaine, and coco ampho glycinate); nonionic surfactant such as alkyl poly(ethylene oxide), or alkyl polyglucosides (octyl glucoside and decyl maltoside), poly(vinyl pyrrolidone) (PVP); and an amphiphilic copolymer such as poly(ethylene glycol)-block-polypropylene glycol)-block-poly(ethylene glycol) (PEO-PPO-PEO).

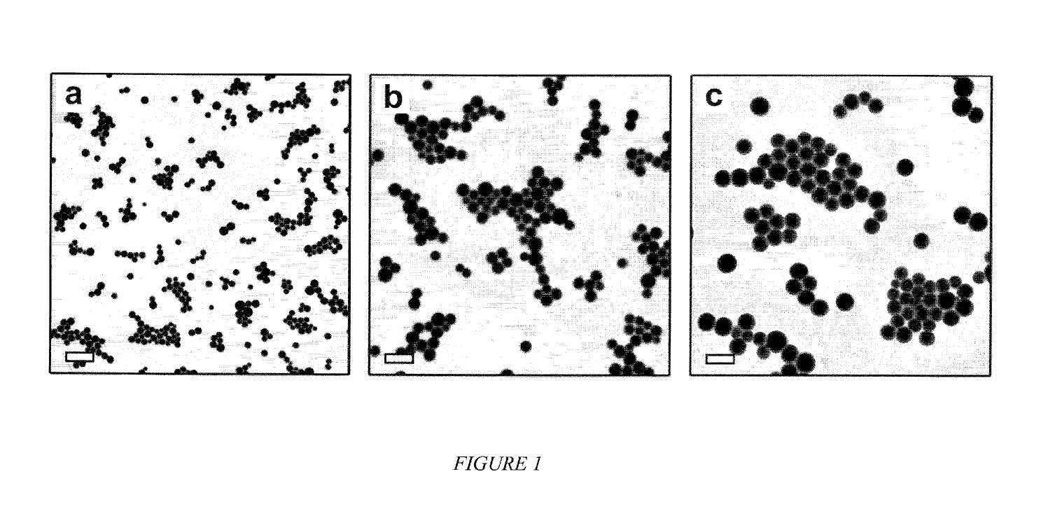

In an exemplary preparation according to an embodiment of the invention, DPPH (0.01 mmol) is dissolved in THF (1 mL) under Ar to form a deep purple-colored stock solution. A 100 μL portion of the stock solution is injected into a flask with 5 mL of water (Nanopure: 18.2 MΩ) at room temperature with vigorous stirring. After a desired growth time (0˜2 hrs) is reached, a gelatin aqueous solution (1.8 mL, wt 2%) is injected into the growth solution. The resulting solution can be stirring until the DPPH NPs are isolated from the growth solution through centrifugation. The resulting NPs are highly dispersible in water. Transmission electron microscopy (TEM) shows that NPs made using different growth times (0 to 2 hours) have diameters ranging from 90 nm to 310 nm with a relative standard deviation of ˜14% as shown in FIG. 1. The DPPH NPs exhibit high stability in water and do not size-ripen over a period of six months or more.

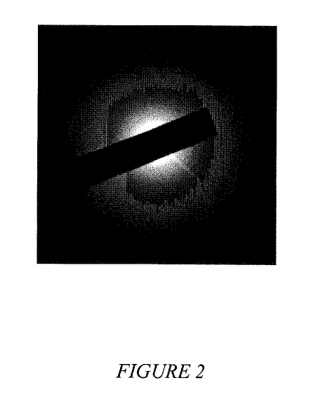

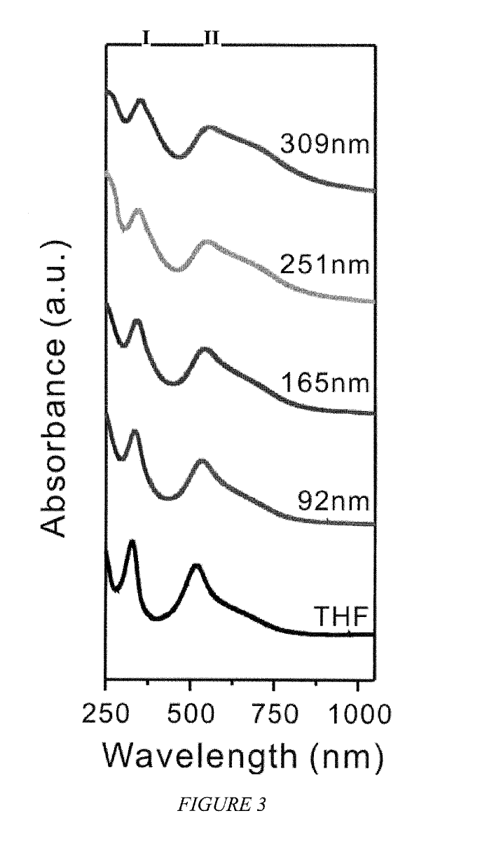

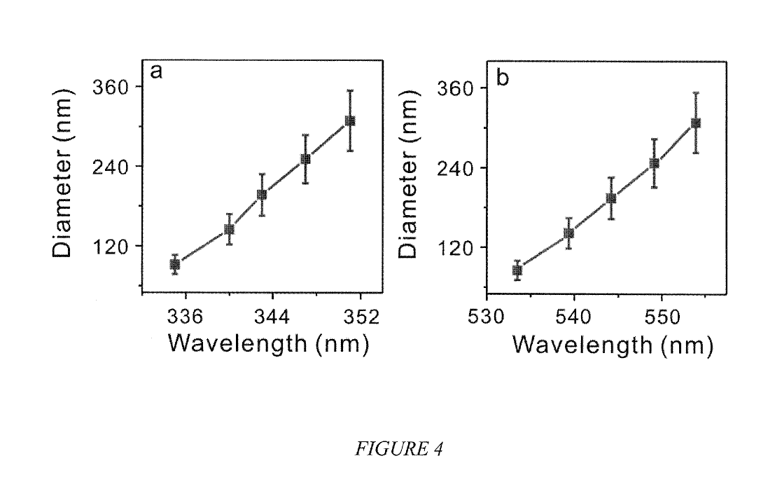

As-prepared DPPH NPs according to an embodiment of the invention show an amorphous structure, as can be seen in FIG. 2. The DPPH NPs display two absorption bands at the UV (I) and visible (II) regime, respectively as shown in FIG. 3. The two bands originate from π-π transitions of the DPPH radical molecule. The delocalized radical electron of DPPH is the major contribution to the visible absorption band (II). Both of the DPPH NPs absorption bands exhibit a size-dependent red shift from the equivalent band for free DPPH molecules in THF, where the larger the NPs, the larger the red shift as can be seen in FIG. 4 for bands I and II. The red shifts for these π-π absorption bands are due to J-type aggregation of DPPH molecules within a NP. The size-dependent red-shift of these bands is consistent with an increase of the intermolecular interactions with increasing size for the DPPH NPs.

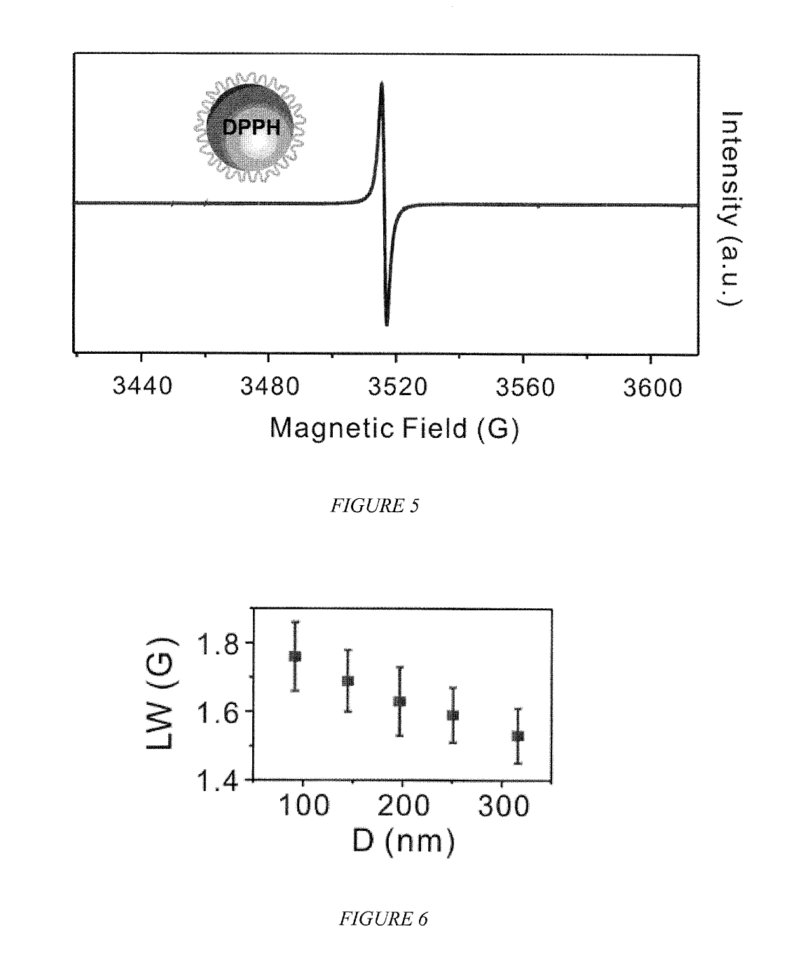

An EPR spectrum of DPPH NPs according to an embodiment of the invention consists of a characteristic single Lorentzian line with a narrow linewidth as shown in FIG. 5. The EPR linewidth weakly depends on the NP's size. As the nanoparticle size decreases from 310 nm to 90 nm, the EPR linewidth increases from 1.50 G to 1.80 G as shown in FIG. 6. The single Lorentzian EPR line and the narrow linewidth of these NPs are due to a fast Heisenberg spin-exchange interaction between DPPH radicals within an individual nanoparticle. The spin-exchange involves a change of spin states of the paramagnetic species that is induced by an exchange interaction between neighboring partners and occurs when these partner's electron orbitals overlap. Depending on specific conditions, the EPR lines of interacting paramagnetic species can be broadened, shifted, or narrowed by spin exchange. Under fast Heisenberg exchange conditions, all the hyperfine EPR lines of individual DPPH radicals merge to a single Lorentzian line whose width linearly decreases with the increase of Heisenberg exchange rate. The slightly broader linewidth of 90-nm DPPH NPs may be due to a slower Heisenberg exchange rate than that of larger NPs. This slower exchange rate may be associated with the weaker intermolecular interaction between DPPH molecules in 90-nm NPs, as indicated by peak positions of their absorption bands, as can be seen in FIG. 3 as plotted in FIG. 4.

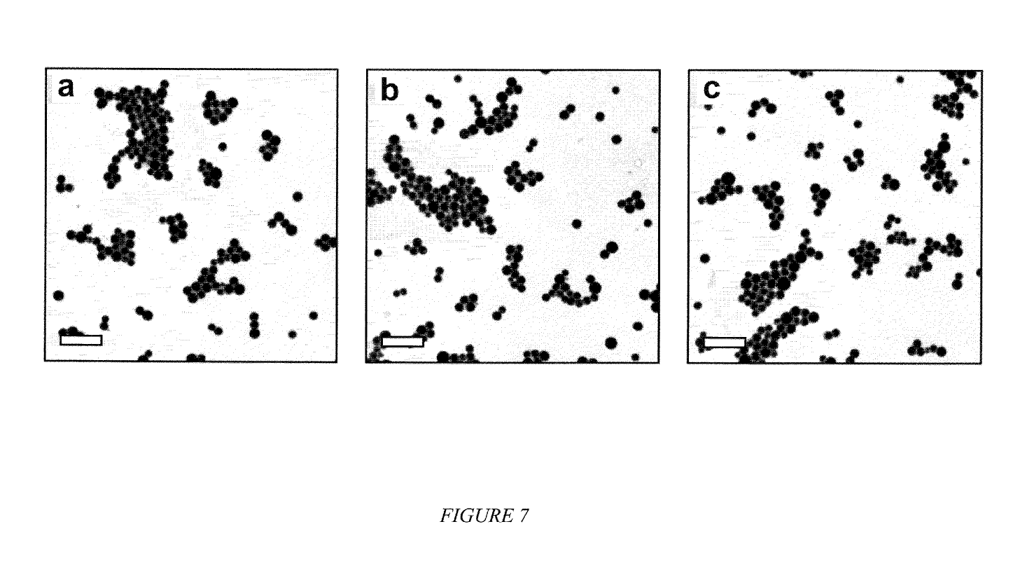

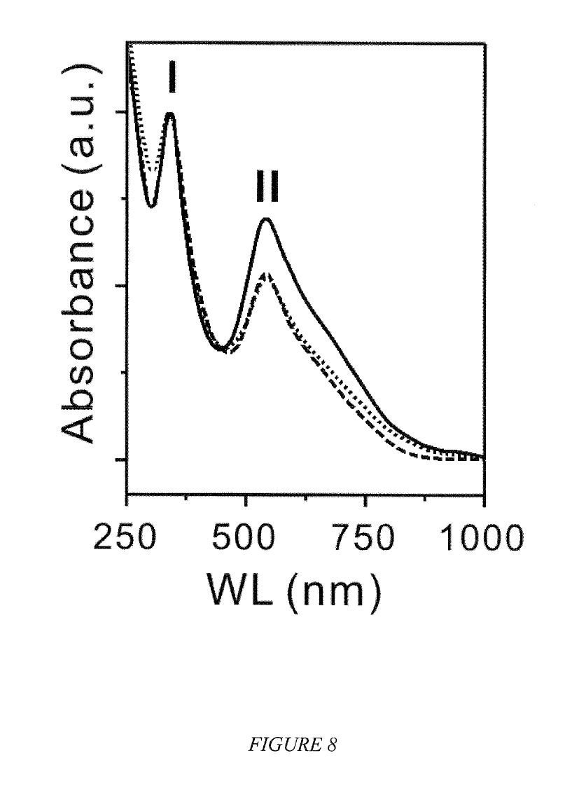

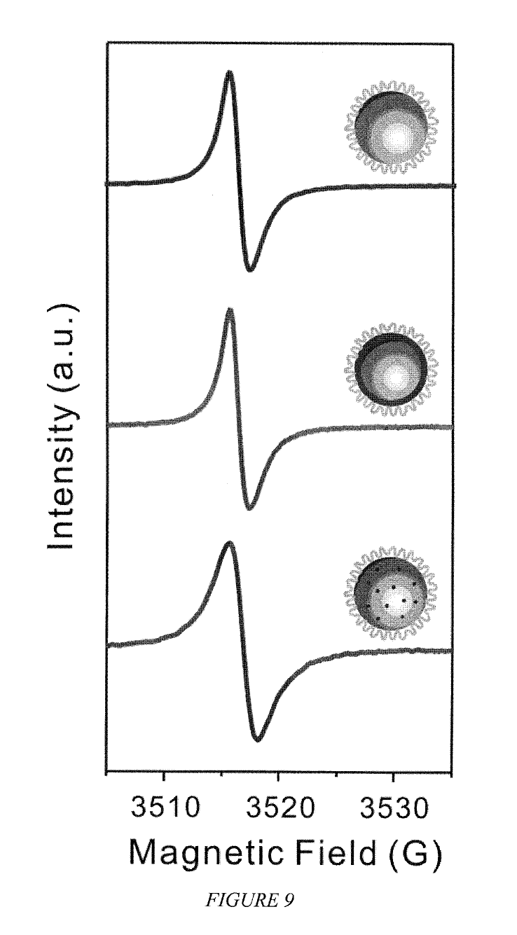

Other embodiments of the invention include a core/shell nanoparticle having a DPPH as the core coated with a shell of 2,2′-diphenyl-1-picrylhydrazine (DPPH-H), a DPPH nanoparticle doped with DPPH-H, and methods to prepare these nanoparticles. DPPH-H is a non-radical, reduced form of DPPH. Without the radical electron, DPPH-H does not have visible band (II) of DPPH, but retains UV band (I) of DPPH. The DPPH, core/shell DPPH/DPPH-H, and DPPH-H doped DPPH nanoparticles differ in their EPR signals. These differences are consistent with a difference in their Heisenberg exchange and the J aggregation of DPPH molecules inside a NP. For example, NPs of nearly identical size, 180 nm, as shown in FIG. 7 for DPPH NPs, core/shell NPs and doped NPs have DPPH-H concentrations of about 20%, exhibit nearly identical reductions in the relative intensity of their visible band (II) relative to the intensity of their UV band (I), as shown in FIG. 8, with no shift from those in their DPPH counterpart, which indicates that DPPH-H does not substantially influence the aggregation of DPPH molecules inside the core/shell or doped NPs. In contrast to the shift and the intensity of absorbance bands, the presence of the DPPH-H dopant significantly affects the NP's EPR linewidth as shown in FIG. 9. As can be seen on examination of FIG. 9, when DPPH-H resides in the shell of a core/shell nanoparticle, an EPR linewidth of 1.70 G, identical to that of the DPPH NPs of similar size, is observed, but when the DPPH-H is doped in the NPs, an EPR linewidth of 2.20 G indicates an approximate 30% reduction in Heisenberg exchange rate from that in the DPPH NPs. This result is consistent with insertion of DPPH-H molecules into the DPPH aggregates and blocking Heisenberg exchange interactions between neighboring DPPH radicals in the nanoparticle. Although Heisenberg exchange interactions have a small impact on the aggregation of DPPH molecules, surface effects do not appear to play a major role in controlling the optical and paramagnetic properties of DPPH NPs.

In other embodiments of the invention paramagnetic nanoparticles other than DPPH, as indicated above, can be used to form doped or core/shell nanoparticles with a diamagnetic dopant or shell. Reducing agents other than 2,5-dihydroxy-1,4-benzoquinone, even hydrogen peroxide, can be employed to form the diamagnetic shell. In other embodiments of the invention NPs with a doped core can be reduced to have a diamagnetic shell.

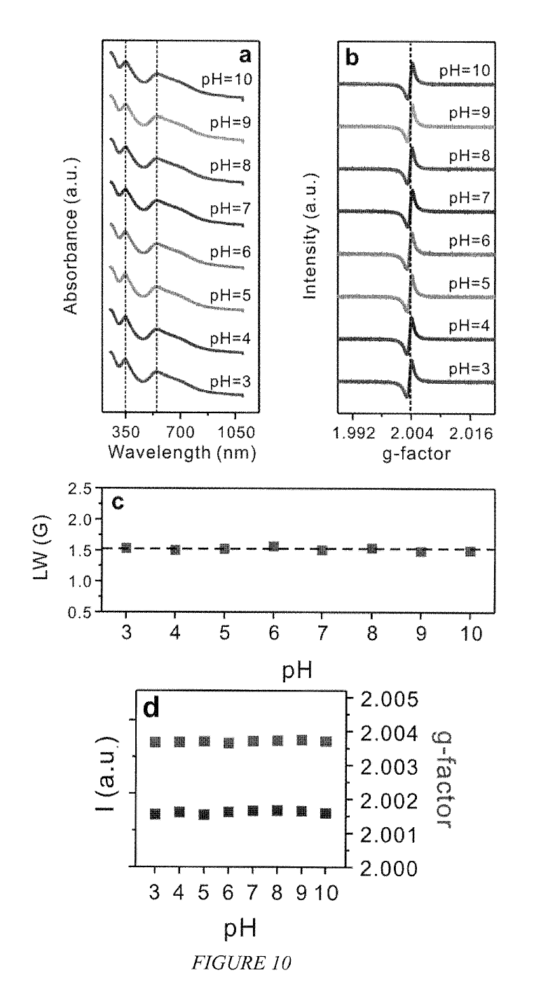

According to an embodiment of the invention, the DPPH comprising NPs can be used as EPR standards. FIG. 10 illustrates that the DPPH NPs are stable over a range of pH. The UV-Vis absorption spectrum, g-factor, EPR linewidth, and intensity of integrated EPR absorption for the DPPH NPs show no measurable variation over a pH range of 3.0 to 10.0. These results indicate that DPPH NPs are stable over a wide pH range and are useful as standard field markers and primary spin-concentration standards in aqueous samples. These novel DPPH NPs are useful for standards for fundamental research and can be used for various applications in the food industry and involving life sciences.

### Methods and Materials

### Chemicals

The compounds: 2,2′-diphenyl-1-picrylhydrazyl (DPPH, free radical), 2,5-dihydroxy-1,4-benzoquinone (DHBQ, 98%), tetrahydrofuran (THF, anhydrous, ≧99.9%), and gelatin (from porcine skin, Type A) were purchased from Aldrich. Nanopure water (18 MΩ cm) was made using a Barnstead Nanopure Diamond system.

### Synthesis of DPPH Nanoparticles

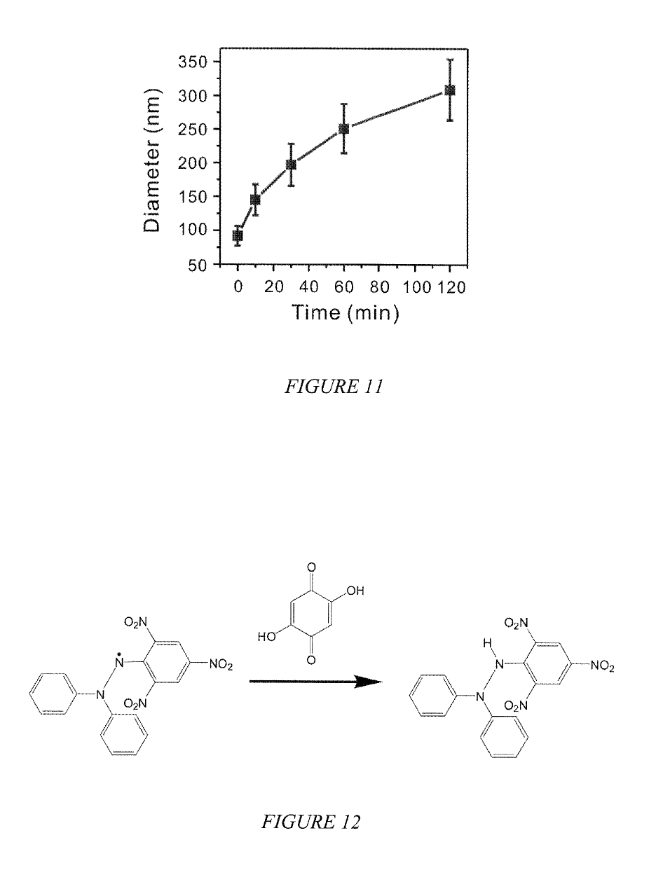

DPPH (0.01 mmol) was dissolved in tetrahydrofuran (THF, 1 mL) under an argon atmosphere to form a deep purple-colored stock solution. A 100 μL portion of this stock solution was injected into nanopure water (18.2 MΩ, 5 mL) at room temperature (25° C.) with vigorous stirring. The growth time was varied over 0 to 2 hours after injecting the DPPH stock solution followed by injection of a 1.8 mL gelatin aqueous solution (2 weight %) into the growth solution with stirring for an additional 5 minutes. For the synthesis of a growth time of 0 hours, 100 μL of DPPH stock solution and 1.8 mL of 2 weight % gelatin aqueous solution were simultaneously injected into a flask with 5 mL of nanopure water (18.2 MΩ), and then the resulting solution was stirred for 5 minutes. In this manner, the final DPPH nanoparticle size depended exclusively by the variation of the growth time as plotted in FIG. 11. In all cases the resulting DPPH nanoparticles were separated from the growth solution by centrifugation, and were subsequently redispersed in nanopure water and centrifuged again.

### Synthesis of DPPH-H-Doped DPPH Nanoparticles

A DPPH-H stock solution was prepared by mixing DPPH (0.01 mmol) and DHBQ (0.01 mmol) in 1 mL of THF, as shown in FIG. 12. This DPPH-H stock solution was mixed with a DPPH stock solution to form stock solutions with DPPH-H/DPPH molar ratios of 2 to 8. A portion (100 μL) of the resulting stock solution was swiftly injected into 5 mL of nanopure water (18.2 MΩ) at room temperature (25° C.) under vigorous stirring. After about 30 minutes of nanoparticle growth, a gelatin aqueous solution (1.8 mL, wt 2%) was injected into the growth solution and the mixture stirred for 5 minutes. The DPPH-H-doped DPPH nanoparticles were separated by centrifuge dispersed in nanopure water, and centrifuged a second time.

### Synthesis of DPPH/DPPH-H Core/Shell Nanoparticles

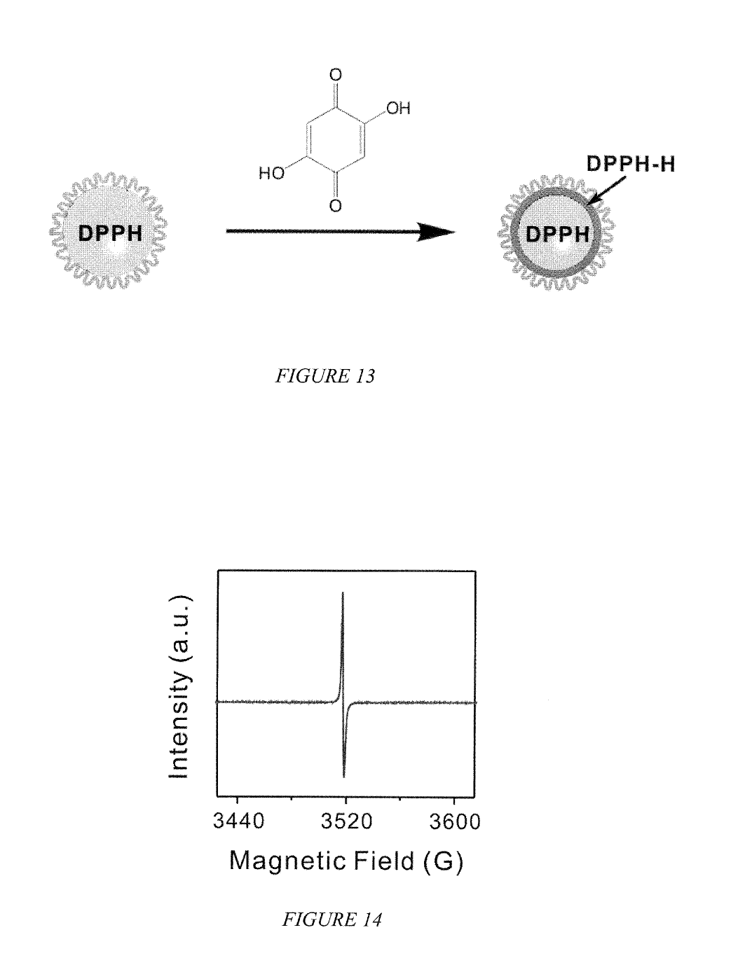

As illustrated in FIG. 13, after synthesis of a DPPH nanoparticle in the manner disclosed above, to yield NPs with an average diameter of 180 nm, 0.03 mmol DHBQ was added into the 6.8 mL original reaction solution to form a 1:30 molar ratio of DPPH:DHBQ, which formed the DPPH/DPPH-H core/shell nanoparticles. The core/shell NPs exhibited a nearly identical absorption spectrum to that of DPPH-H-doped DPPH nanoparticles according to the above procedure. Then the resulting DPPH/DPPH-H core/shell nanoparticles were purified by centrifugation and recentrifugation, as described above.

Synthesis of DPPH Nanoparticles with a Very Narrow EPR Linewidth

DPPH (0.01 mmol) was dissolved in tetrahydrofuran (THF, 1 mL) under argon protection to form a deep purple-colored stock solution. 100 μL of this stock solution was injected into deionized (DI) water (5 mL) at room temperature (25° C.) with vigorous stirring. After nanoparticle growth for about 60 minutes, a gelatin aqueous solution (1.8 mL, wt 2%) was injected into the growth solution and the mixture stirred for 5 minutes. The resulting DPPH nanoparticles were separated by centrifuge, redispersed in DI water, and recentrifugation. By using water of much lower conductivity, the EPR linewidth, as shown in FIG. 12, of the resulting DPPH nanoparticles narrowed to 1.18G which is less than the narrowest linewidth (1.5G) of common DPPH standards that are in the form of water-insoluble microcrystals.

### TEM and ED Measurements

TEM measurements were performed on a JEOL 200X operated at 200 kV, or a JEOL 2010F TEM operated at 200 kV. ED measurements were acquired by the 2010F TEM and operated at 200 kV. The specimens were prepared as follows: a particle solution (10 μL) was dropped onto a 200-mesh copper grid, and was dried overnight at ambient conditions.

### EPR Measurements

EPR measurements were performed at room temperature in CW mode on an X-band Bruker Elexsys 580 spectrometer (9.5 GHz) using an Oxford ESR900 cryostat.

### EPR Simulations

Raw EPR data were imported into the 2D plotting and data analysis tool “Grace.” The spectra were first baseline-corrected and then fitted to a Lorentzian line shape using the non-linear curve fitting tool with Formula 1 for the differentiated Lorentzian function where A is the amplitude, ΔB is the linewidth, and B_{R} is the resonance field.

$$\frac{d P}{d B} = - 2 A \frac{Δ B \times ( B - {B}_{R} )}{{( Δ {B}^{2} + {( B - {B}_{R} )}^{2} )}^{2}} \qquad Formula 1$$

The electronic g-factor for each transition was determined from its resonance field using Formula 2 for the resonance condition:

$$g = \frac{h v}{{μ}_{B} {B}_{R}} \qquad Formula 2$$

where h is Planck's constant and μ_{B} is the Bohr magneton.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A water soluble paramagnetic nanoparticle comprising 2,2′-diphenyl-1-picrylhydrazyl (DPPH) with a diameter of 5 to 1,000 nm.

2. The nanoparticle of claim 1, wherein said nanoparticle is amorphous.

3. The nanoparticle of claim 1, further comprising 2,2′-diphenyl-1-picrylhydrazine (DPPH-H).

4. The nanoparticle of claim 3, wherein said DPPH-H comprises a dopant in said DPPH.

5. The nanoparticle of claim 3, wherein said DPPH-H comprises a shell about a DPPH core.

6. An aqueous electron paramagnetic resonance (EPR) standard comprising a plurality of water soluble paramagnetic nanoparticles according to claim 1.

7. The EPR standard of claim 6, wherein said line width is equal to or less than 1.80 G.

8. The EPR standard of claim 6, wherein said line width is equal to or less than 1.50 G.

9. The EPR standard of claim 6, wherein said line width is equal to or less than 1.18 G.

10. A method for preparation of a water soluble paramagnetic nanoparticle comprising the steps of:
providing an organic solution comprising a paramagnetic organic compound and a water miscible organic solvent;
injecting said organic solution into agitated water to form a growth solution;
maintaining said agitation of said growth solution for a period of time wherein a plurality of nanoparticles of said paramagnetic organic compound grow in size;
injecting an aqueous surfactant solution into said growth solution, wherein said plurality of said nanoparticles size is fixed by said period of time; and
isolating said plurality of said nanoparticles.

11. The method of claim 10, wherein said paramagnetic organic compound comprises DPPH.

12. The method of claim 10, wherein said water miscible organic solvent comprises tetrahydrofuran.

13. The method of claim 10, wherein said water is agitated by rapid stirring.

14. The method of claim 10, wherein said period of time is about 1 minute to about 2 hours.

15. The method of claim 10, wherein said surfactant comprises gelatin.

16. The method of claim 10, wherein said step of isolation comprises centrifuging.

17. The method of claim 10, wherein said organic solution further comprises a diamagnetic compound.

18. The method of claim 17, wherein said diamagnetic compound comprises DPPH-H.

19. The method of claim 10, further comprising the step of injecting a reducing agent after the step of injecting said aqueous gelatin solution, wherein reduction of an outer portion of said nanoparticles forms a core/shell nanoparticle.

20. The method of claim 19, wherein said reducing agent comprises 2,5-dihydroxy-1,4-benzoquinone, said nanoparticles comprise DPPH, and said core/shell nanoparticles comprise DPPH-H/DPPH.
